(19) United States
(12) Patent Application Publication
(10) Pub. No.: US 2004/0228435 A1
(43) Pub. Date: Nov. 18, 2004

(54) Phantom for intensity modulated radiation therapy

(76) Inventor: Kevin J. Russell, melbourne, FL (US)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/438,303

(22) Filed: May 14, 2003

(51) Int. Cl. A61B 6/00

(52) U.S. Cl. 378/18

(57) ABSTRACT

Disclosed is a phantom for dose verification for intensity-modulated radiation therapy having a base of substantially tissue-equivalent material and a two-dimensional array of cavities formed in the base with each the cavities being configured and dimensioned to receive a radiation detector.

### BACKGROUND OF THE INVENTION

1. Field of the Invention

This invention relates to a phantom for dose characterization of IMRT beams.

2. Description of Related Art

Radiation therapy devices for the treatment of tumors in patients, using radiation emitting devices, are well known. A radiation therapy device generally includes a gantry which can be swiveled around a horizontal axis of rotation in the course of a therapeutic treatment. A linear accelerator is located within the gantry for generating a high energy radiation beam for therapy. This high energy radiation beam may be an electron beam or photon (x-ray) beam, for example. During treatment, the radiation beam is trained on a zone of a patient lying in the isocenter of the gantry rotation.

In order to control the radiation emitted toward the patient, a beam shielding device, such as a plate arrangement or collimator, is typically provided in the trajectory of the radiation beam between the radiation source and the patient. The beam shielding device defines a treatment field on the patient for which a prescribed amount of radiation is to be delivered. The usual treatment field shape results in a three-dimensional treatment volume which includes segments of normal tissue, thereby limiting the dose that can be given to the tumor. The goal of radiation therapy is to deliver a high, curative dose to a tumor, while minimizing the dose to normal tissues and limiting the dose in critical healthy structures to their radiation dose tolerance.

Sculpting the beam profile is accomplished using a technique referred to as intensity modulated radiation therapy (IMRT). The essence of IMRT techniques is to vary the characteristics of the radiation therapy beam in real time, while the radiation treatment is actually taking place. These characteristics that are varied include spatial (geometrical), energy, and temporal parameters.

With IMRT, there are many steps between the calibration of the beam of the therapy radiation unit to the determination of the radiation dose at the desired point of interest in the patient. The alignment of the radiotherapy simulators and treatment machines must be checked regularly to maintain accurate localization and treatment. Comprehensive quality assurance tool are used in order to verify any planned treatments, so that the absolute dose delivered (measured dose) is consistent with the planned or prescribed dose. In radiation therapy, it is important to ensure that the absolute dose delivered is consistent with the planned dose, and that the critical spatial resolution of that dose is consistent with the planned dose distribution.

The verification of IMRT patient treatment dosages typically is accomplished with dose measurement phantoms. The phantom simulates the body tissue and utilizes dosimeters to measure the radiation dosage before the treatment process on the patient is commenced. Conventional phantoms have limited versatility.

U.S. Pat. No. 6,364,529 discloses a phantom which provides multiple locations throughout the entire phantom for placement of dosimeters, so as to enable a clinician to evaluate high dose gradient areas, inhomogeneity regions, and dose distribution at sensitive structures. A product advertisement for an 91230 IMRT Dose Verification Phantom from Standard Imaging, entitled “IMRT Dose Verification Phantom, describes a chamber phantom slab with six cavities for ion chamber placement for absolute dose verification in multiple locations throughout the phantom. An IMRT/3D QA Phantom, manufactured by MED-Tech, discloses a phantom with absolute dose verification in multiple locations throughout the phantom. Generally, many of these designs have multiple cavities for the use of multiple radiation detectors. These designs do not provide the flexibility of locating a radiation detector for point dose verification at locations throughout the treatment field of the beam.

### SUMMARY OF THE INVENTION

The present invention is directed toward a phantom for dose verification in intensity-modulated radiation therapy, comprising a base of substantially tissue-equivalent material and a two-dimensional array of cavities formed in said base with each said cavity being configured and dimensioned to receive a radiation detector.

One advantage of the phantom in accordance with the present invention over the prior art designs is the provision of a point dose Quality Assurance (QA) phantom which contains a two-dimension matrix or array of ion chamber positions for flexibility in selecting measurement points. This flexibility allows for more precise sampling of the IMRT radiation field needed in order to verify individual treatment plans.

### BRIEF DESCRIPTION OF THE DRAWINGS

FIG. 1 is a perspective view of an IMRT phantom in accordance with the present invention.

FIG. 2A is a front planar view of an IMRT phantom in accordance with the present invention.

FIG. 2B is a top planar view of an IMRT phantom in accordance with the present invention.

FIG. 2C is a side planar view of an IMRT phantom in accordance with the present invention.

FIG. 3A is a first part of IMRT Absolute Dose QA Form used in accordance with the present invention.

FIG. 3B is a second part of IMRT Absolute Dose QA Form used in accordance with the present invention.

FIG. 4A is a first part of a screen display of treatment planning software used with the present invention.

FIG. 4B is a second part of a screen display of treatment planning software used with the present invention, which shows images of the cavities of the IMRT phantom superimposed over the treatment plan.

FIG. 5 is an IMRT radiation therapy device in which the phantom of the present invention may be used.

### DETAILED DESCRIPTION OF PREFERRED EMBODIMENTS OF THE INVENTION

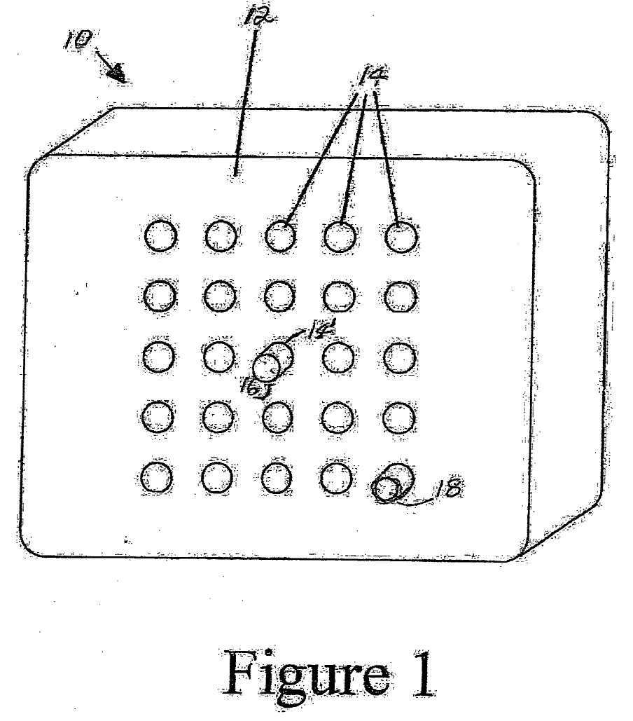

Referring now to the drawings, preferred embodiments of the invention will be described. In FIG. 1, there is shown a phantom 10 in accordance with the present invention. The phantom 10 includes a base 12, which contains a two-dimensional, rectangular array or matrix of cavities 14 formed in the base 12. Each cavity 14 is dimensioned and configured for having a radiation detector inserted therein. For the purposes of illustration, a single radiation detector 16, preferably in the form of an ion chamber, is shown inserted in the center cavity 14′. In a preferred embodiment, the two-dimensional array has an odd-by-odd number of cavities 14 so as to provide a central cavity 14′. Preferably, the cavities 14 do not extend all the way through the base 12 of the phantom 10. Although a ion chamber is the preferred radiation detector, those skilled in the art will recognized that other radiation detectors may be used with the present invention, such as solid state detectors, e.g., diode detectors.

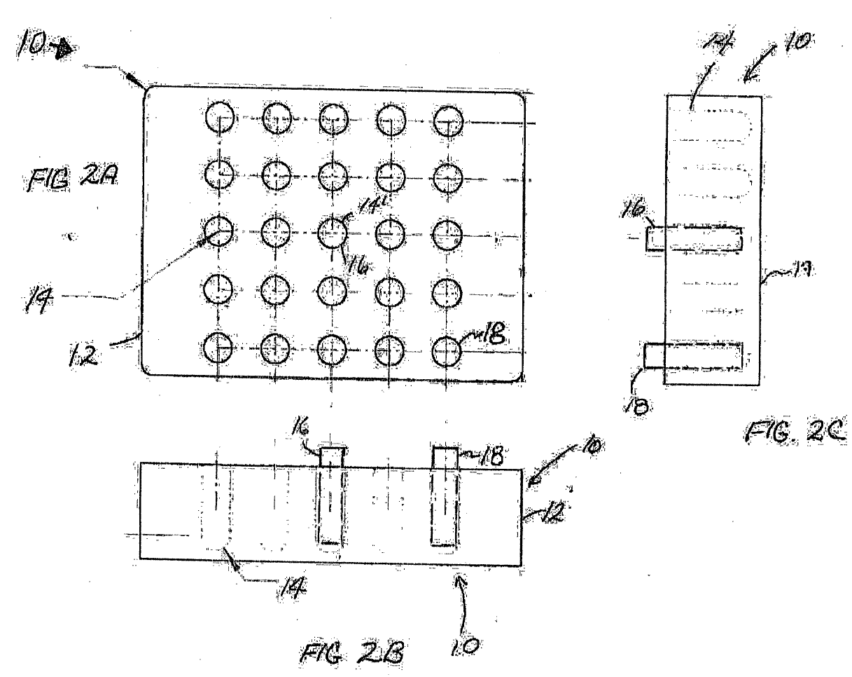

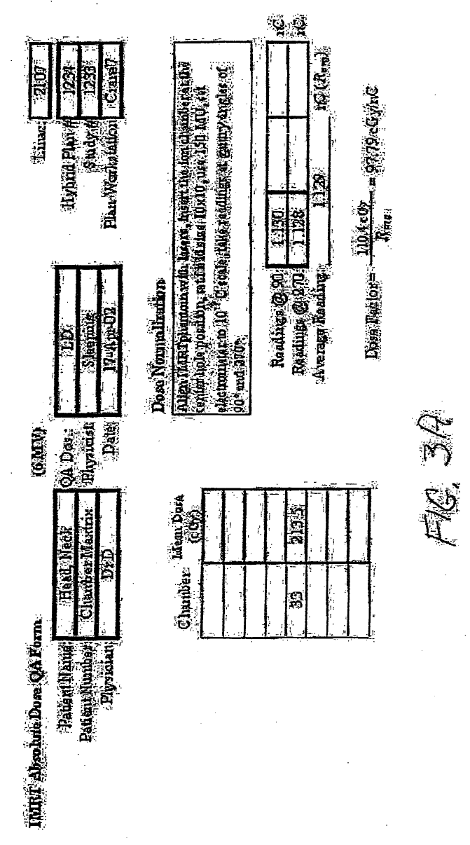

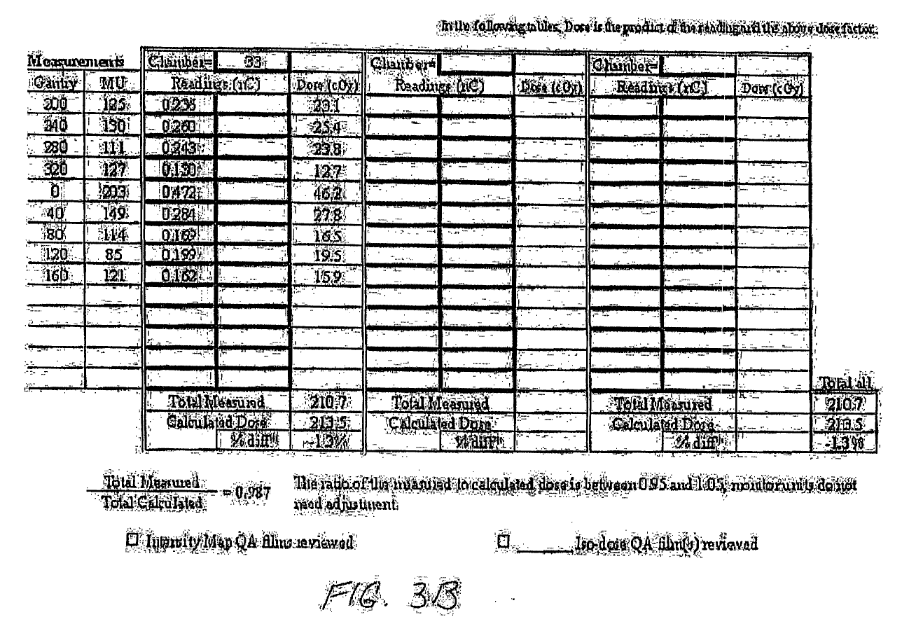

Referring to FIGS. 2A-2C, the cavities 14 are adapted in dimension to receive-a radiation detector 16, although normally only one cavity at a time will have a radiation detector, as will be explained hereinafter. As will discussed hereinafter, when in use, the cavities 14 of the phantom that do not include a radiation detector 16 will contain a plug 18 (only one shown in FIGS. 2A-2C) made out of the same material as the base 12. In the preferred embodiment, the diameter for a cavity 14 is 14.3 cm with the cavities 14 spaced about 3 cm or less apart. In the preferred embodiment, there is an array of five-by-five cavities 14, with the width and height of the base 12 generally being about 15 cm to about 25 cm, which are useful dimensions for calibrating most linacs. However, depending upon the linac, different sizes may be desirable, since the number of cavities and spacing between cavities is somewhat a function of the size of the treatment field. The phantom 10 need not be square, but may be rectangular as shown in the drawings. The overall thickness of the phantom will generally be from 3 to 15 cm, preferably about 5 cm. In the embodiment shown, the base 12 is about 6 cm thick between at the ends of the cavities 14 and the rear wall 19 (see FIG. 2C) of the base 12. Generally, the base 12 is sized so as to provide enough radiation scatter material and the desired equivalent depth into water (which simulates human tissue). The comers are rounded to provide a smooth contour, which is better for the software fitting.

A preferred material for manufacturing the phantom 10 of the invention is crosslinked polystyrene, such as that commercially sold under the trademark Rexolite™. Useful materials for the phantom 10 include any materials commonly used in medical physics as water-equivalent or close water-equivalents, such as Solid Water™ sold by RMI and others. Rexolite has the advantage of being machined relatively easily and also has a cost advantage. Generally, any material that is a near tissue/water equivalent under the high energy radiation to be measured is suitable for use with the invention.

An IMRT phantom calibration procedure, in accordance to the present invention, is described in detail hereinafter. This calibration procedure is used for comparing a planned IMRT radiation dose calculated by a treatment planning system (“calculated dose”) with the dose actually delivered by a radiation beam in linac under ideal calibration conditions (“measured dose”), so as to provide a comparison between the calculated dose and the measured dose. In the preferred embodiment, the treatment planning system or CT-simulator may comprise Corvus or Pinnacle 3D planning software or like planning application software. As used hereinafter, “commissioning” is the term used when a new IMRT machine is brought on line: installed, adjusted, tested, etc . . . to assure that it is functioning within operational parameters. “Patient QA (quality assurance)” is verification that the patient is receiving the right amounts of radiation dose and at the required location(s).

As an overview, the phantom calibration procedure may be viewed as having two calibration stages. There is a first calibration using a radiation detector and a water tank as the phantom during commissioning when the phantom is in a “flat phantom condition”. There is a second calibration using the phantom of the present invention, when the phantom is in a “in phantom condition”. This second calibration process may be repeated every few months or on an “as needed” basis and is independent of any particular patient case. Both calibrations will be described in detail hereinafter.

After the radiation detector has been calibrated, the linac is characterized as being in a “linac calibration condition”. With a new patient case, a Quality Assurance (QA) measurement process is initiated using the phantom in accordance to the present invention (“QA measurement process”), wherein the actual delivered dose at a given point is measured and compared with the planned dose. This QA measurement process implemented on the phantom provides the necessary quality assurance to ensure that a subsequent treatment will go as planned when actually delivered to the patient.

The setup for the first calibration process, using the water tank, is as follows. First, during commissioning, a water tank is used as a phantom in deriving calibration parameters and conversion factors. To make measurement easier, motorized 1-D water tank is ideal for this purpose. An ion chamber is mounted horizontally and move vertically at different depths. A CNMC 1-D motorized water tank is used, model RMD-100-3. Capintec 1D water phantom (Item # 5250-0103) may also be used. A “flat phantom” geometry is used, i.e., a gantry angle of 0 degrees. The user sets up the measurement condition for this linac calibration during the commissioning process. For example, the MD Anderson Cancer Center in Houston, Tex. (MDACC) typically uses 100 cm SSD (source-to-surface-distance) at Dmax (depth into tissue or material where the radiation reading is maximum of each beam), which would typically calibrate to about 1.0 cGy per MU. One cGy is exactly equal to one ‘rad’ of radiation. MU stands for ‘Monitor Units’—the linac has a monitor that tells the user the amount of radiation delivered.

The setup for the second calibration process, using the phantom of the present invention, is as follows. The phantom is positioned within the linac. The phantom is preferable centered at isocenter of the linac using conventional methods, such as lasers. The isocenter is the center position of the radiation treatment. The conventional use of lasers typically involves having three laser beam coming in from different orthogonal directions so that they intersect at right angles to each other, with the lasers typically being mounted into the walls and ceilings. After being positioned in the linac, a CT-scan of the phantom is made and the resulting CT-scan image is transferred to the treatment planning system. Preferably, the whole phantom should be included in the CT scan. CT image slices are taken of the phantom, generally at a slice spacing of from about 1 to 5 mm, preferably less than or equal to 3 mm, preferably at about 2 mm at a thickness substantially equal to the spacing so as to achieve good spatial resolution. It is preferred that the entire height, width and depth of the phantom be so scanned, so as to enable the effective use of the phantom in measuring vertex beams (beams not perpendicular to the phantom's surface) that enter the phantom's front surface. Absent a complete image, these vertex beams may be entered at an incorrect source-to-surface-distance (SSD) in the treatment planning software. It is also preferable to place a detector in one of the cavities 14 so as to make the outline of the detector volume easier to see in the CT images.

Using the results of both of the above described calibration processes, an equation will be derived for calculating the dose actually delivered by a radiation beam in linac under ideal calibration conditions (“measured dose”). The reading for a detector may be calibrated in terms of a dose factor (DF) that is defined as:

$$DF ≡ \frac{CDO \times {MU}_{flat}}{{TPC}_{flat} \times {R}_{flat}} = \frac{{Dose}_{flat}}{{TPC}_{flat} \times {R}_{flat}} ( cGy / Rdg ) \qquad ( 1 )$$

where R_{flat} is the detector reading in the “flat phantom” setup geometry, MU_{flat} is the MU delivered to the flat phantom, and TPC_{flat} is the temperature and pressure correction factor in the flat phantom calibration condition. Equation 1 assumes the Calibrated Dose Output (CDO) to be equal to 1 cGY/MU in the flat phantom calibration condition, therefore 150 cGy after correction for temperature and pressure was delivered to the calibration point. The dose conversion factor (DCF) is then given by:

$$DCF = DF ( cGy / Rdg ) = \frac{{Dose}_{flat}}{{TPC}_{flat} \times {R}_{flat}} {( cGy / Rdg )}^{2} \qquad ( 1 b )$$

Using the above water tank under calibration conditions is not practical for daily patient QA. Hence, the calibration condition of Equation 1 needs to be transferred to a setup condition that is suitable for patient QA measurement on a daily basis. Hence, this creates the need for the second calibration described above using the phantom according to the present invention. The second calibration may be performed using the phantom to derive a Transfer Factor (TF) to relate the readings of Equation 1 to an “in-phantom” calibration geometry. As mentioned above, he phantom is aligned to the isocenter of the scanned images by aligning to lasers and then taking radiation readings preferably, but not necessarily at two different gantry angles. For a typical linac, jaw openings of the collimator would be set to just equal or less than the dimensions of the array of detector cavities 14. Hence, for a 13.5 cm by 13.5 cm array, such as disclosed in the drawings, the jaw opening might be set at about 10 cm×10 cm or so. Again, a radiation detector is placed in the center cavity 14′, which is aligned with the center of the jaw opening. The electrometer of the dosimeter will typically be set to 10⁻⁹ C scale. The two gantry angles will generally be at least about 45° apart, preferably at least about 90° apart, still more preferably about 180° apart. Typical gantry angles for taking the radiation readings are at 90° and 270° at a delivered monitor unit (MU) of 150. Preferably, several reading will be taken to generate average readings at each angle, which are then used to compute an average overall reading, R_{avg}:

$${R}_{avg} = \frac{\overline{{R}_{90}} + \overline{{R}_{270}}}{2} ( Rdg ) \qquad ( 2 )$$

where R₉₀ and R₂₇₀ are the average radiation readings at gantry angles 90° and 270°, respectively.

By using the same radiation detector as was used in the previous flat geometry calibration, we may calculate the dose factor (DF) as:

$$DF = \frac{TF}{{TPC}_{IMRT} \times {R}_{avg}} ( cGy / Rdg ) \qquad ( 3 )$$

where TF is the transfer factor that transfers radiation detector calibration from the flat phantom geometry to the in-phantom calibration condition. TPC_{IMRT} is the temperature and pressure correction for the phantom under IMRT radiation conditions.

Occasionally, the water temperature in the calculation of Equation 1 for the in-water calibration is different than for the in-phantom temperature. Because both the flat phantom and the in-phantom measurements were performed at the same time, Equations 1 and 3 are combined to derive the transfer factor (TF):

$$TF = {TPC}_{IMRT} \times {R}_{avg} \times DF = \frac{{TPC}_{IMRT} \times {R}_{avg} \times {Dose}_{flat}}{{TPC}_{flat} \times {R}_{flat}} ( cGy ) \qquad ( 4 )$$

where R_{avg} is from Equation 3. Take note that Equation 4 assumes the same MU was delivered for both calibrations. The transfer factor (TF) is a constant for a given ion chamber and a given treatment beam.

Once the transfer factor (TF) is derived from the commissioning process, the dose to any point measured in the phantom during a routine patient QA measurement may be calculated by:

$${Dose}_{p} = DF \times {TPC}_{IMRT} \times {\sum }_{i = 1}^{n} {R}_{i} = \frac{TF}{{R}_{avg}} \times {\sum }_{i = 1}^{n} {R}_{i} ( cGy ) \qquad ( 5 )$$

where Rᵢ is the radiation detector reading for the i^{th} treatment field and n is the total number of treatment fields (typically at different gantry angles) for the particular treatment plan.

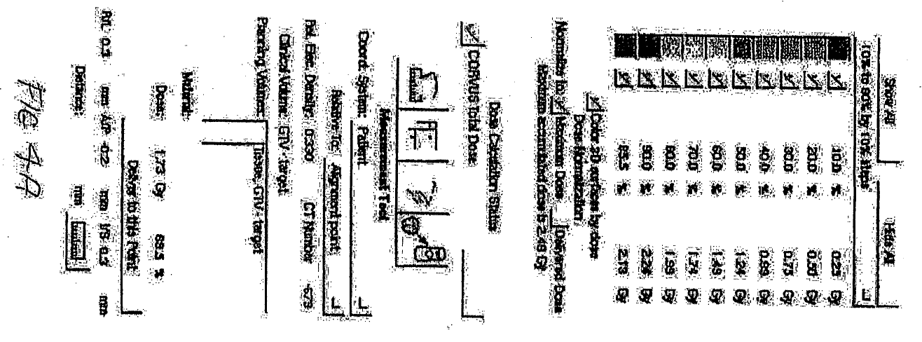

FIGS. 3A and #B shows an IMRT Absolute Dose QA worksheet form (divided over the two FIGS) which is completed by the therapist during the QA measurement process. Under the box labeled “Dose Normalization”, the instructions for the above described second calibration process are provided, which include “Align IMRT phantom with lasers, insert the ion chamber at the center hole position, set field size 10×10, use 150 MU, set electrometer to 10⁻⁹ C scale, take readings at gantry angles of 90° and 270°”. Referring back to Equation 2, R₉₀ and R₂₇₀ are the average radiation readings at gantry angles 90° and 270°. Hence, in FIG. 3A, the reading at gantry angles 90° and 270° are 11.30 nC and 11.28 nC, respectively. Using Equation 2, R_{avg} is equal to 1.29 nC. Using equation #3, the Dose Factor (DF) is calculated to be 97.79 cGy/nC.

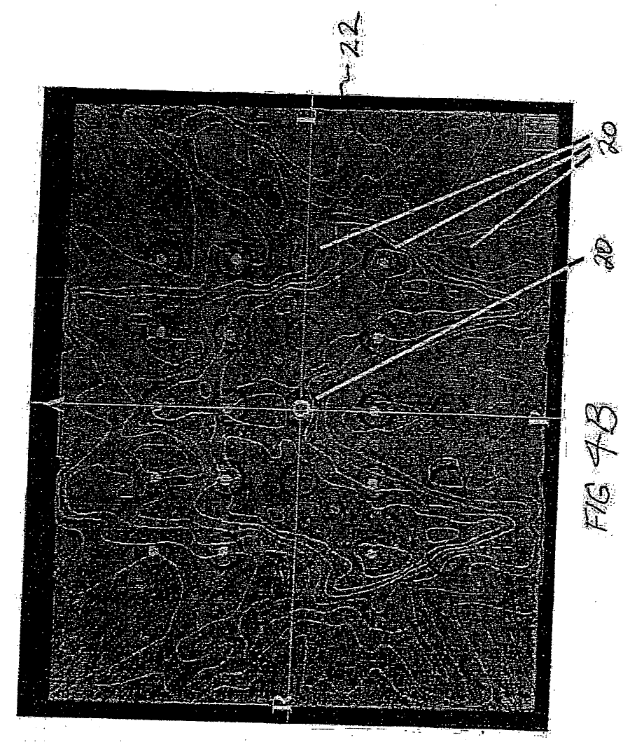

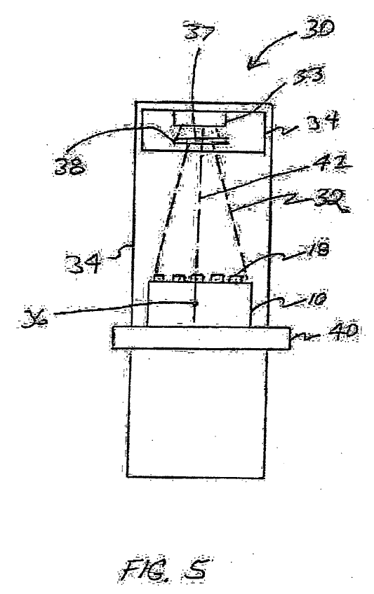

With the completion of the two linac calibration processes, the QA measurement process for IMRT MU verification for a given patient can be undertaken. In the following brief summary, the improved QA measurement process of the present invention includes the following steps: (1) locating the point of interest for the IMRT MU verification, (2) preferably, but not necessarily, move the center of the phantom to this point, (3) generate the hybrid phantom QA plan, (4) locate a cavity position for measurement. Finally, use these results to conduct a measurement. This QA measurement process is described in more detail with respect to FIGS. 3A, 3B, 4A and 4B.

Referring to FIGS. 3A and 3B, the sample dose measurement and worksheet is shown. This is a spreadsheet for cavity calibration and dose measurements. As an example of an IMRT MU verification process, a head and neck case, using the Corvus planning software, is selected for illustration. In a conventional manner, the treatment planning software generates a CT-scans (not shown) of the area of concern (location of tumor) from three directions, along with a planned dose. Typically, an Isodose line distribution is provided, with this distribution being superimposed over the CT scans, which in this illustrated case is of the head and neck of the patient. The first step of this QA measurement process is for the therapist to use these CT-scans and Isodose line distributions to select a location of interest. Typically, the therapist writes down the location of the point of interest (for example, R/L −0.6 mm, A/P 5.4 mm, and I/S 45.0 mm—three dimensional coordinates in this illustrative case, provided by the Corvus treatment planning software). Next, the therapist preferably, but not necessarily, moves the center of the phantom to this selected point.

The next step is for the therapist to generate a hybrid phantom plan. The Corvus planning software has an easy procedure to move the phantom isocenter (which is at (0,0,0) in its own coordinate system) to a new point of interest in the original plan (−0.6, 5.4, 45.0, in this illustrative hybrid plan). The hybrid phantom QA plan is then calculated.

FIGS. 4A and 4B illustrates the hybrid phantom QA plan by showing the screen display of the planning software, but split between two Figures. In FIG. 4A the there is a color coded Isodose line display. In FIG. 4B these lines are mapped over a two dimensional space. Images 20 of the cavities of phantom are superimposed over this Isodose display, with the Isodose lines being shown by numeral 22. The therapist reviews the calculated hybrid phantom QA plan and locates one of the 25 cavity positions for measurement. It is desirable to place the ion chamber in the low gradient location of the field as well as the high dose region such as the center of the targets. For example, referring to FIG. 4B, if the therapist wants a dose verification measurement in a high dose region, the Isodose lines specify where those high dose regions are (Isodose lines are color coded to specify Gy amounts as shown in the chart of FIG. 4A). If the therapist also wants the measurement to be taken in a low gradient region, for the reasons discussed herein, then the therapist will select one of the cavity images in the high dose regions, but which is not located in an area having a high concentration of Isodose lines.

Referring back to FIG. 3B, there is illustrated an application of Equation 5 for computing the total delivered Doseₚ, which is calculated to be 210.7 cGy. This is compared with the planned (calculated) dose of 213.5 cGy, to give a difference of −1.3%. In this illustrative case, the ion chamber was positioned in chamber (cavity) 33, which is the center cavity. Each row represents one of the treatment fields (each at a different gantry), with n=9 in this illustrative example. In accordance with Equation 5, the radiation dose of each treatment field is summed to get the total measured dose.

FIG. 5 shows an illustrative, conventional IMRT radiation therapy device 30 in which the phantom 10 of the present invention may be used. A radiation beam 32 from a radiation source 33 is shown being delivered to the phantom 10. This beam 32 is produced by the linear accelerator (“linac”) which is mounted in a gantry 34. The gantry 34 can rotate about a 360 degree arc around an isocenter 36. Each beam 32 is shaped by an opening 37 in a collimator 38, such opening 37 typically being defined by one or more pairs of jaws, in a conventional manner. The phantom 10 is positioned on a rotatable table 40. The gantry 34 and table 40 both rotate about the isocenter 36. A center line 42 of the beam 32 defines the gantry angle of the gantry 34, with the vertical position shown in FIG. 5 representing a zero degree gantry angle. Typically, the center line 42 of the beam 32 also passes through the isocenter 36. The beam 32, after being shaped by the collimator 38, defines a treatment field on the phantom 10 which is the area subjected to radiation. As described above, for a typical linac, the opening 37 of the collimator 38 would be set to just equal or less than the dimensions of the array of detector cavities 14. Hence, for a 13.5 cm by 13.5 cm array of cavities, such as disclosed in the drawings, the jaw opening might be set at about 10 cm×10 cm. It should be noted that in this comparison, we are comparing the cross section of the opening 37 with the cross section of the array of cavities of phantom 10 and that these two cross sections are substantially parallel to each other and both cross sections are substantially perpendicular to the center axis 42 of the radiation beam 32. It also should be noted from FIG. 5 that the beam 32 does spread somewhat after leaving the collimator 38, so that the cross sectional area of the beam when it intercepts the phantom 10 is greater than the cross sectional area of the beam after leaving the collimator 38. In general, it is desirable that the cross sectional area defined by the outer cavities of the array of cavities to be equal to or greater than the cross sectional area of the field of treatment for the patient.

One advantage of ion cavity matrix phantom of the present invention is that it is very easy to pick different ion chamber positions for measurement without the need of re-calculating a hybrid plan. Even though the ion chamber used here is very small, it's still a good idea to avoid measuring in a rapid changing dose gradient region. This is because a small setup error can cause a relatively large dosimetric difference in IMRT measurement. In other words, one advantage of the phantom in accordance with the present invention over the prior art design is the provision of a point dose Quality Assurance (QA) phantom which contain two-dimension matrix or array of ion chamber positions for flexibility in selecting measurement points. This flexibility allows for more precise sampling IMRT radiation field needed in order to verify individual treatment plans.

It is important to select the best possible point for conducting a dose verification. Having 25 locations to choose from (5×5 matrix) offers the user the flexibility to select this best point. This array of the phantom has cavities separated by 3 cm so your best resolution is 3cm. Specifically, the user wants to select a point that is in a low-gradient region. This means that the user wants to select a point at a location where being a little off to the left or to the right does not represent a large amount of change. Rather, the user wants to select a location where the radiation level is “flat”—i.e., being off to the left or right leaves what is being measured essentially unchanged. Placing the radiation detector at this point is optimum.

Another advantage of the present invention is that the single piece construction of the phantom allows easy setup for in-room measurement and, as described above, reduces the effort of generating separate phantom calculation plans. This is in contrast to the existing prior art practice in which selection of the measurement position is achieved by offsetting the phantom positions by a pre-determined amount during measurement.

While various values, scalar and otherwise, may be disclosed herein, it is to be understood that these are not exact values, but rather to be interpreted as “about” such values, Further, the use of a modifier such as “about” or “approximately” in this specification with respect to any value is not to imply that the absence of such a modifier with respect to another value indicated the latter to be exact.

Changes and modifications can be made by those skilled in the art to the embodiments as disclosed herein and such examples, illustrations, and theories are for explanatory purposes and are not intended to limit the scope of the claims.

What is claimed is:

1. A phantom for dose verification in intensity-modulated radiation therapy, comprising:
a base of substantially tissue-equivalent material; and
a two-dimensional array of cavities formed in said base with each said cavity being configured and dimensioned to receive a radiation detector.

2. The phantom according to claim 1, wherein each dimension of said two-dimensional array has an odd number of said cavities so as to define a center cavity.

3. The phantom according to claim 2, further including:
at least one radiation detector being disposed in one of said cavities, and
a plug of substantially tissue-equivalent material being disposed in all of said cavities not containing one of said radiation detectors.

4. The phantom according to claim 3, wherein said cavities are spaced apart from each other by no more than 3 centimeters.

5. The phantom according to claim 3, wherein said array of cavities is dimensioned to be equal to or greater than a field of treatment used in the intensity-modulated radiation therapy.

6. A radiation therapy device for intensity-modulated radiation therapy, comprising:
a radiation source providing a radiation beam;
a collimator having an opening for shaping said radiation beam;
a phantom for dose verification of said radiation beam;
said phantom having a base of substantially tissue-equivalent material and a two-dimensional array of cavities formed in said base, with each said cavity being configured and dimensioned to receive a radiation detector; and
said opening of said collimator 38 having a cross section dimensioned to be equal to or less than a cross section of said two-dimensional array of cavities.

7. The radiation therapy device in accordance with claim 6, wherein said cross section of said opening is substantially parallel to said section of said two-dimensional array and both said cross sections are substantially perpendicular to a center axis of said radiation beam.

8. The radiation therapy device according to claim 6, wherein each dimension of said two-dimensional array has an odd number of said cavities so as to define a center cavity.

9. The radiation therapy device according to claim 8, further including:
at least one radiation detector being disposed in one of said cavities, and
a plug of substantially tissue-equivalent material being disposed in all of said cavities not containing one of said radiation detectors.

10. A method of using a phantom for radiation dose verification in a field of treatment of an intensity modulated radiation therapy device, comprising the steps of:
providing a phantom with a two-dimensional array of cavities, each said cavity being dimensioned and configured to receive a radiation detector;
positioning said phantom in said field of treatment;
generating at least one treatment plan which includes an image of said field of treatment with a plurality of isodose lines for the said radiation dose;
superimposing an image of said array of cavities over said treatment plan; and
selecting one of said cavities for placement of said radiation detector.

11. The method according to claim 10, wherein said step of selecting one of said cavities is based upon the disposition and concentration of said isodose lines relative to a cavity image of said selected cavity, said cavity image being part of said image of said array in said treatment plan.

12. The method according to claim 11, wherein said step of selecting one of said cavities includes selecting one of said cavity images of said treatment plan that is in a low gradient area of said isodose lines.

13. The method according to claim 10, further including the step of:
scanning said phantom to generate said image of said array of cavities, said image of said array comprising a CT scan of said phantom.

14. The method according to claim 11, wherein said step of generating said treatment plan further includes the steps of generating a CT-scan of the patient and superimposing said isodose lines over said CT-scan of said patient.

15. The method according to claim 14, further wherein said step of generating at least one treatment plan includes generating an original treatment plan and a hybrid treatment plan and said step of selecting one of said cavities for placement of said radiation detector being based upon said hybrid treatment plan.

16. The method according to claim 15, further including the steps of:
locating a first point of interest in said original treatment plan;
moving the center of said phantom to said first point of interest;
generating said hybrid treatment plan based upon a new point of interest; and
superimposing said CT scan of said array over said hybrid treatment plan.
